(19) United States
(12) Patent Application Publication
(10) Pub. No.: US 2026/0102254 A1
(43) Pub. Date: Apr. 16, 2026

(54) SHOULDER IMPLANT

(71) Applicant: Malhas Medical Limited, Eastbourne (GB)

(72) Inventor: Amar MALHAS, Eastbourne (GB)

(21) Appl. No.: 19/359,051

(22) Filed: Oct. 15, 2025

(30) Foreign Application Priority Data
Oct. 15, 2024 (GB) 2415120.1

(51) Int. Cl. A61F 2/40; A61F 2/30

(52) U.S. Cl. CPC A61F 2/4081; A61F 2/30734; A61F 2/30749; A61F 2/30942; A61F 2/4059; A61F 2002/4085

(57) ABSTRACT

The present invention relates to designing a reverse shoulder implant for use in a human as part of a shoulder replacement surgical procedure. It will optimise post operative function and can be used to design future implants.

### FIELD OF THE INVENTION

The present invention relates to designing a reverse shoulder implant for use in a human as part of a shoulder replacement surgical procedure. It will optimise post operative function and can be used to design future implants.

### BACKGROUND TO THE INVENTION

Shoulder replacements are routinely performed for human patients for a variety of reasons, including arthritis, cuff arthropathy and trauma. A shoulder replacement sees all or part of the glenohumeral joint replaced with an implant. Historically, “anatomic” shoulder replacements were widely used. The humeral head would be removed and replaced with a prosthetic implant comprising a metal ball/hemisphere (the humeral head) provided on the end of a stem. The stem is attached within the humeral shaft. The shoulder socket (the glenoid) is modified through attachment of a plastic socket against which the new humeral head rotates. Hence, the shoulder anatomy is replicated in metal and plastic.

More recently, reverse shoulder implants have become popular. In a reverse shoulder implant, the locations of the ball and socket are reversed.

That is, an implanted hemisphere (the new ball and so called “glenosphere”) is attached to the glenoid, and a dished liner (the new socket) is attached to the humerus via a stem instead. Thus the normal shoulder geometry is reversed.

Rotation of the implanted shoulder sees the liner rotate around the glenosphere, as driven by the deltoid muscle and stabilised primarily by the tendons of the rotator cuff and secondarily by the deltoid muscle.

The success of such reverse shoulder implants depends on selecting suitable design variables for the implant components. These design variables include the size of the glenosphere and its lateralisation relative to the glenoid, and the location and orientation of the liner on the humerus. Using implants with sub-optimal design variables can lead to a lateral shift in the humeral position which, in turn, leads to incorrect tensioning of the deltoid muscle. If the deltoid is under-tensioned, the shoulder will tend to dislocate. Conversely, over-tensioning the deltoid at best leads to stiffness and loss of mobility, and at worst can see acromial fracturing. A further problem is notching where the lower part of the liner can butt up against the lower part of the scapula when the arm is in the relaxed position by the patient's side.

As a result, over time various designs have developed to reduce these risks. Many will change dimensions such as the glenosphere size, and the lateral offset (how far away the humeral shaft is held from the glenoid and hence how well tensioned the muscles are). There are over 30 designs on the market with varying offsets and component sizes. The challenge arises in matching those components to a human patient. Human beings range from the very small to the very large and while most implants cater for the “average” person, there is recognition that people on the extreme ends of the spectrum are harder to treat. There is also great debate as to how to optimise function with no agreement on how much offset is needed or which components should provide the offset.

Hence, there is a need for a method of designing a reverse shoulder implant and identifying a set of design variables for the set of implant components that both reduces the risk of surgery as well as gives the patient an optimal outcome that is designed around their specific dimensions.

### SUMMARY OF THE INVENTION

A method of designing a reverse shoulder implant is described that may be bespoke to each specific patient, and seeks to reproduce the native joint's offset. This may be done using standard components to reproduce the native joint's offset.

The use of reverse shoulder implants leads to a difference in how the shoulder operates. In a native shoulder, the point about which the humerus rotates is known as the centre of rotation (COR). The centre of rotation is located within the humeral head, and remains static such that the distance from the native glenoid to the centre of rotation remains the same whichever direction the arm is moved. In a reverse implant, the centre of rotation is now at the centre of the glenosphere near the glenoid. The most lateral point of the glenosphere can be defined as the pole of the glenosphere. When the arm is held at rest by the body or is raised forwards (forward flexion), the liner contacts the glenosphere towards a mid-hemispherical point. During forward flexion, the contact point between the liner and glenosphere follows a latitudinal plane of the glenosphere (i.e. rotates around the pole such that the lateral offset of the point of contact relative to the glenoid remains static).

Since the arm is at rest, we define this as the arm in the 0 degrees position.

When the arm is raised to the side (abduction), the liner travels across the glenosphere in a longitudinal plane (i.e. the point of contact moves up towards the pole, across the pole, and then continues its upward direction of travel which is now away from the pole). Hence, the lateral offset of the point of contact between the liner and the glenosphere is no longer static and varies as the liner travels over the pole of the hemisphere. When the arm is raised to the side to be horizontal, it is at 90 degrees of abduction and so we term this the 90 degree position of the arm. Accounting for this change in lateral offsets in addition to the average lateral offset leads to better patient outcomes.

According to a first aspect, the present invention resides in a method of designing a reverse shoulder implant. The shoulder implant comprises implant components that include a glenosphere for attachment to a patient's glenoid and a dished liner for attachment to a patient's humerus. The dish of the liner receives the glenosphere when the implant is in place in the patient.

The method comprises determining a native total lateral offset of the native shoulder. The total native lateral offset corresponds to the lateral offset of the centre of rotation position in the native humeral head relative to a native glenoid reference point.

The method also comprises using a set of design variables of the implant components to determine a total lateral offset of the implanted shoulder. The 0 degrees total lateral offset corresponds to the lateral offset of a reference point on the liner from the native glenoid reference point when the implanted shoulder is in 0 degrees alignment (e.g. with the humeral shaft axis of the arm extending vertically). The set of design variables of the implant components are also used to determine the 90 degrees total lateral offset of the implanted shoulder. The 90 degrees total lateral offset corresponds to the lateral offset of the reference point on the liner from the native glenoid reference point when the implanted shoulder is in a 90 degrees alignment (e.g. with the humeral shaft axis extending horizontally in abduction).

The 0 degrees and 90 degrees total lateral offsets relative to the native total lateral offset are then displayed. The 0 degrees and 90 degrees total lateral offsets describe the range of lateral offset (delta offset) the implant goes through as the arm travels from rest to differing degrees of forward flexion and abduction. Optionally, the average of the 0 degrees and 90 degrees total lateral offsets relative to the native total lateral offset is also displayed.

This allows comparison of the different lateral offsets. The scientific hypothesis is that: the better a patient's native lateral offset is reproduced by the new implant, the better the shoulder muscles will be tensioned and the better the post-operative functional outcome and range of movement will be. It has been appreciated that patient outcomes are statistically improved by matching the 0 degrees and 90 degrees total lateral offsets to each other and also to the native total lateral offset. Matching the 0 degrees and 90 degrees total lateral offsets to the patient's original native lateral offset leads to a well balanced shoulder. Matching the 0 degrees and 90 degrees total lateral offsets to the native total lateral offset provides better tensioning of the deltoid muscle, and so reduces the risk of stiffness and instability.

Determining the native total lateral offset may comprise determining the native total lateral offset as the radius of the humeral head. This is advantageous as determining the centre of rotation position is difficult without templating software, whereas measuring the size of the humeral head is relatively straightforward. A better approximation may be achieved by multiplying the radius by a factor between 0.95 and 1, for example 0.98. This reflects the fact that the humeral head represents ⅘^{th} of the hemi-sphere of the centre of rotation. The radius of the native humeral head may be measured from an image taken of the patient's native shoulder, for example an image obtained using computer tomography (CT) or x-rays.

The native glenoid reference point may be determined from an image taken of the patient's native shoulder, for example an image obtained using computer tomography or x-rays. The native glenoid reference point may be taken as the point where the native humeral head contacts the native glenoid in an intact shoulder joint. This may require an approximation due to arthritic wear and erosion in the glenoid moving the glenoid reference point in the worn glenoid (the “neo glenoid”) away from where the glenoid reference point would be in an intact shoulder. To determine the native glenoid reference point, Friedman's axis may be used. Friedman's axis is a well-defined line passing laterally through the scapula from the medial scapula border and that exits the scapula at the centre of the intact glenoid. In the presence of significant arthritis and glenoid erosion, the native glenoid reference point can be determined using the neo glenoid and an approximation of where Friedman's axis would continue and pass through the intact native glenoid.

The liner may comprise a reverse side and an obverse side that is provided with the dish. The liner reference point may be taken to correspond to the position on the reverse side directly beneath the centre of the dish. More formally, the liner reference point may be taken to be the point where a line drawn perpendicularly through the centre of the dish intersects the reverse side. Then, the set of design variables may include the thickness of the liner which corresponds to the separation of the liner reference point from the centre of the dish.

Optionally, the method further comprises determining the 0 degrees total lateral offset to include an adjustment corresponding to the lateral offset of the liner reference point transverse to the humeral shaft axis relative to the lateral offset of the centre of the native humeral head transverse to the humeral shaft axis. Also, the 90 degrees total lateral offset may be determined to include an adjustment corresponding to the on-axis offset of the liner reference point along the humeral shaft axis relative to the position of the centre of the native humeral head along the humeral shaft axis. Then, the set of design variables of the implant components may include the lateral offset of the liner reference point transverse to the humeral shaft axis and the on-axis offset of the liner reference point along the humeral shaft axis. This is advantageous as it accounts for a change in the native humerus as a result of implanting the liner: movement of the liner reference point relative to the humeral shaft axis relative to the position of the centre of the humeral head may be taken into account.

The lateral offset of the centre of the native humeral head transverse to the humeral shaft axis may be determined from an image taken of the patient's native shoulder. When specifying the design variables of the implant components, the lateral offset of the liner reference point transverse to the humeral shaft axis may be specified to be equal to the lateral offset of the centre of the native humeral head transverse to the humeral shaft axis. This mimics the native shoulder, and so provides a good baseline for the initial design, even if the lateral offset of the liner reference point is modified later.

Determining the 0 degrees and 90 degrees total lateral offsets of the implanted shoulder may comprise determining the lateral offset of the centre of the dish of the liner from the native glenoid reference point, determining the lateral offset across the liner, and adding the lateral offset of the centre of the dish of the liner from the native glenoid reference point to the lateral offset across the liner. This may provide a convenient way of determining the total lateral offsets trigonometrically.

Determining the lateral offset of the centre of the dish of the liner from the native glenoid reference point may comprise determining the lateral offset of a glenosphere reference point from the native glenoid reference point. The glenosphere may comprise a part-sphere and the glenosphere reference point may coincide with the centre of the corresponding complete sphere. Then, the lateral offset across the glenosphere may be determined from the glenosphere reference point to the centre of the dish of the liner. Finally, the lateral offset of the glenosphere reference point from the native glenoid reference point may be added to the lateral offset across the glenosphere.

Optionally, determining the lateral offset of the glenosphere reference point from the native glenoid reference point comprises determining the lateral distance from the glenosphere reference point to the attachment point where the glenosphere is attached to the bone of the glenoid less the lateral length of bone loss between the native glenoid reference position and the attachment point. The set of design variables of the implant components may include the lateral length of bone loss between the native glenoid reference position and the attachment point. This accounts for the position of the glenosphere as attached to the glenoid relative to the native glenoid reference position, and may be used when determining both the 0 degrees total lateral offset and the 90 degrees total lateral offset. In addition to bone loss in the native shoulder relative to an intact shoulder due to bone erosion, the glenoid may need to be reshaped and so the lateral bone loss resulting from this reshaping is also taken into account as this would otherwise cause a further medialisation of the shoulder joint.

Design variables of the implant components may cause a lateralisation of the shoulder joint. For example, the glenosphere may be provided on a baseplate, either as an integral unit or joined to the baseplate. In this case the baseplate is interposed between the glenosphere and the glenoid, and so the lateral thickness of the baseplate may be included in the distance from the glenosphere reference point to the attachment point. Rather than attaching the glenosphere of baseplate directly to the glenoid, the attachment may be made indirectly via a lateralising peg. As the lateralising peg is interposed between the glenosphere and the glenoid, the lateral length of the lateralising peg extending between the glenosphere reference point and the attachment point may be included in the distance from the glenosphere reference point to the attachment point. The lateralising peg may be bare or may support a bone graft to fill the gap between the glenoid and the glenosphere/baseplate.

The set of design variables may include the radius of the glenosphere and the neck angle of the liner. The neck angle may be equal to the angle between the normal to the centre of the dish of the liner and the humeral shaft axis. When determining the 0 degrees total lateral offset, determining the lateral offset across the glenosphere from the glenosphere reference point to the centre of the dish of the liner may comprise calculating the lateral offset across the glenosphere as the radius of the glenosphere times the cosine of a tilt angle equal to the neck angle minus 90 degrees. When determining the 90 degrees total lateral offset, determining the lateral offset across the glenosphere from the glenosphere reference point to the centre of the dish of the liner comprises calculating the lateral offset across the glenosphere as the radius of the glenosphere. This arises from the natural positioning of the glenosphere relative to the liner in the 90 degrees position: the centre of the dish aligns with the lateral edge of the glenosphere such that the lateral separation is equal to the radius of the glenosphere.

When specifying the design variables of the implant components, the glenosphere radius may be specified to be equal to the native humeral head radius. This places the centre of rotation in the implanted shoulder in approximately the same place as for the native shoulder, and so provides a good baseline for the initial design, even if modified later. The native humeral head radius may be measured from an image taken of the patient's native shoulder, for example from a CT scan or x-ray.

When determining the 0 degrees total lateral offset, determining the lateral offset across the liner may comprise calculating the lateral offset across the liner as the thickness of the liner times the cosine of the tilt angle.

Optionally, the liner may include a lateralisation component in which case the set of design variables of the implant components may include a liner lateralisation value. Some manufacturers have optional liners with built in lateralisation and some have lateralisation built into all their liners. Then, when determining the 0 degrees total lateral offset, determining the lateral offset across the liner may further comprise adding the liner lateralisation value to the calculated value of the thickness of the liner times the cosine of a tilt angle when determining the lateral offset across the liner at 0 degrees. When determining the 90 degrees total lateral offset, determining the lateral offset across the liner may comprise calculating the lateral offset across the liner as the thickness of the liner times the sine of the tilt angle.

Advantageously, the methods described above may be employed as part of an iterative design process. The design variables may be modified to see what difference they have on the 0 degrees and 90 degrees total lateral offsets relative to the native total lateral offset. In this way, a set of design variables may be found that provide the best matched 0 degrees and 90 degrees total lateral offsets and/or the 0 degrees and 90 degrees total lateral offsets best matched to the native total lateral offset.

Hence, the set of design variables of the implant components referred to above may be an original set of design variables of the implant components.

The method may then further comprise generating an updated set of design variables of the implant components by modifying one or more of the design variables of the original set of design variables. The updated set of design variables may be used to determine updated 0 degrees and 90 degrees total lateral offsets. The updated 0 degrees and 90 degrees total lateral offsets may be presented relative to the native total lateral offset.

Modifying one or more of the design variables may comprise modifying one or more of the glenosphere radius, the thickness of the glenosphere baseplate, the lateral length of the lateralising peg extending between the glenosphere reference point and the attachment point, lateral length of bone loss between the native glenoid reference position and the attachment point, the thickness of the liner, the lateralisation of the liner, the lateral offset of the liner reference point transverse to the humeral shaft axis, the on-axis offset of the liner reference point along the humeral shaft axis, and the neck angle of the liner.

The method may further comprise presenting the average of the updated 0 degrees and 90 degrees total lateral offsets relative to the native total humeral offset. Also, the method may further comprise presenting the updated 0 degrees and 90 degrees total lateral offsets relative to the original 0 degrees and 90 degrees total lateral offsets.

The method may comprise one or more further iterations of the steps of generating updated sets of design variables of the implant components by modifying one or more of the design variables of the original set of design variables or an updated set of design variables; using each updated set of design variables to determine updated 0 degrees and 90 degrees total lateral offsets; and presenting each of the updated 0 degrees and 90 degrees total lateral offsets relative to the native total lateral offset. The method may then further comprise selecting one set of design variables of the implant components from the initial set of design variables and the updated sets of design variables. The selection may comprise selecting the set with the smallest difference between the average of the 0 degrees and 90 degrees total lateral offsets and the native total humeral offset and/or the smallest difference between the 0 degrees and 90 degrees total lateral offsets.

The design variables of the original set of design variables may be set by a person. Alternatively, the original set of design variables may be set by a suitably-programmed computer system. For example, the design variables of the original set of design variables may be determined by a computer system using an analysis of historical data that comprise design variables for previously performed reverse shoulder implants. The computer system may use machine learning to identify an optimum set of design variables to use as the original set of design variables.

The design variables of the updated sets of design variables may be set by a person. For example, the display of the 0 degrees and 90 degrees total lateral offsets relative to the native total lateral offset may be reviewed by a surgeon who modifies the design variables using skill and judgement.

Alternatively, the design variables of the updated sets of design variables may be determined by a suitably-programmed computer system. For example, the computer system may employ an optimisation routine to modify the design variables, and may use a brute force method or a guided algorithm method.

The computer system may use a cost function to score the original 0 degrees and 90 degrees total lateral offsets relative to the native total lateral offset and the updated 0 degrees and 90 degrees total lateral offsets relative to the native total lateral offset, and select the set of design variables achieving the best score.

The present invention also resides in a computer program comprising computer program instructions that, when executed by one or more computer processors, cause the one of more computer processors to perform any of the methods described above, or a computer readable medium having such a computer program stored thereon. Furthermore, the present invention resides in a computer system comprising one or more computer processors and a computer memory having stored therein a computer program comprising computer program instructions that, when executed by the one or more computer processors, cause the one of more computer processors to any of the methods described above.

According to a second aspect, the present invention resides in a set of implant components configured for a reverse shoulder implant. The set of implant components comprises a glenosphere assembly, a humeral stem and a liner. The glenosphere assembly includes a glenosphere and a baseplate for attaching the glenosphere to the glenoid of a patient. The humeral stem is configured for implanting into the humerus of the patient. The dished liner is fastened to or configured to be fastened to the humeral stem. The design variables of the set of implant components are such that the 0 degrees and 90 degrees total lateral offsets of the implanted shoulder match the total lateral offset in the patient's native shoulder. The total lateral offset of the native shoulder corresponds to the lateral offset of the centre of rotation position in the native humeral head relative to a native glenoid reference point. The 0 degrees total lateral offset of the implanted shoulder corresponds to the lateral offset of a reference point on the liner from the native glenoid reference point when the implanted shoulder is in a 0 degrees alignment with the humeral shaft axis of the arm extending vertically. The 90 degrees total lateral offset of the implanted shoulder corresponding to the lateral offset of the reference point on the liner from the native glenoid reference point when the implanted shoulder is in a 90 degrees alignment with the humeral shaft axis extending horizontally.

According to a third aspect, the present invention resides in a reverse shoulder implant comprising a glenosphere assembly, a humeral stem and a liner. The glenosphere assembly includes a glenosphere and a baseplate that attaches the glenosphere to the glenoid of a patient. The humeral stem is implanted into the humerus of the patient. The dished liner is fastened to part of the humeral stem. The design variables of the components of the reverse shoulder implant are such that the 0 degrees and 90 degrees total lateral offsets of the implanted shoulder match the total lateral offset in the patient's native shoulder. The total lateral offset of the native shoulder corresponds to the lateral offset of the centre of rotation position in the native humeral head relative to a native glenoid reference point. The 0 degrees total lateral offset of the implanted shoulder corresponds to the lateral offset of a reference point on the liner from the native glenoid reference point when the implanted shoulder is in a 0 degrees alignment with the humeral shaft axis of the arm extending vertically. The 90 degrees total lateral offset of the implanted shoulder corresponding to the lateral offset of the reference point on the liner from the native glenoid reference point when the implanted shoulder is in a 90 degrees alignment with the humeral shaft axis extending horizontally.

According to the second and third aspects of the invention, the native total lateral offset may correspond to the radius of the native humeral head multiplied by a factor between 0.95 and 1, for example 0.98. The native glenoid reference point may correspond to the point where Friedman's axis crosses the native glenoid.

The liner may comprise a reverse side and an obverse side that is provided with the dish. The dish may have a thickness which corresponds to the distance between the centre of the dish and a liner reference point. The liner reference point thus corresponds to the position on the reverse side beneath the centre of the dish. More formally, the liner reference point may correspond to the point where a line drawn perpendicularly through the centre of the dish intersects the reverse side.

The liner may be positioned on the humerus to give a 0 degrees total lateral offset that includes an adjustment corresponding to the lateral offset of the liner reference point transverse to the humeral shaft axis relative to the lateral offset of the centre of the native humeral head transverse to the humeral shaft axis. Also, the liner may be positioned on the humerus to give a 90 degrees total lateral offset that includes an adjustment corresponding to the on-axis offset of the liner reference point along the humeral shaft axis relative to the position of the centre of the native humeral head along the humeral shaft axis.

The 0 degrees and 90 degrees total lateral offsets of the implanted shoulder may include the lateral offset of the centre of the dish of the liner from the native glenoid reference point plus the lateral offset of the centre of the dish of the liner from the native glenoid reference point to the lateral offset across the liner. The lateral offset of the centre of the dish of the liner from the native glenoid reference point may correspond to the lateral offset across the glenosphere plus the lateral offset of a glenosphere reference point from the native glenoid reference point. The glenosphere may comprise a part-sphere and the glenosphere reference point may coincide with the centre of the corresponding complete sphere. Then, the lateral offset across the glenosphere may correspond to the glenosphere reference point to the centre of the dish of the liner, and hence is dependent upon the size of the glenosphere. The lateral offset of the glenosphere reference point from the native glenoid reference point may correspond to the lateral distance from the glenosphere reference point to the attachment point where the glenosphere is attached to the bone of the glenoid less the lateral length of bone loss between the native glenoid reference position and the attachment point. The glenosphere assembly may comprise a baseplate with a certain thickness and, optionally, a lateralising peg separating the glenosphere from the baseplate. The lateral offset of the glenosphere reference point from the native glenoid reference point may include the thickness of the baseplate and the separation of the baseplate and glenoid reference point as set by the lateralising peg.

The humeral stem may be configured to set a neck angle of the liner. The neck angle may be equal to the angle between the normal to the centre of the dish of the liner and the humeral shaft axis. The neck angle contributes to the total lateral offsets of the implanted shoulder. For the 0 degrees total lateral offset, the lateral offset across the glenosphere from the glenosphere reference point to the centre of the dish of the liner will comprise the radius of the glenosphere times the cosine of a tilt angle equal to the neck angle minus 90 degrees. For the 90 degrees total lateral offset, the lateral offset across the glenosphere from the glenosphere reference point to the centre of the dish of the liner is in fact just the radius of the glenosphere. Moreover, for the 0 degrees total lateral offset, the lateral offset across the liner will comprise the thickness of the liner times the cosine of the tilt angle. For the 90 degrees total lateral offset, the lateral offset across the liner will comprise the thickness of the liner times the sine of the tilt angle.

### LIST OF FIGURES

In order that the invention can be more readily understood, reference will now be made by way of example only, to the accompanying drawings in which:

FIGS. 1A and 1B are views of a native glenohumeral joint showing the deltoid and rotator cuff respectively;

FIGS. 2A and 2B are computer generated representations of a glenohumeral joint showing a native humerus and clavicle with the arm relaxed in a 0 degrees orientation and with the arm slightly raised in approximately 30 degrees orientation respectively;

FIG. 3A shows a native humeral head and FIG. 3B shows a native humeral head and glenoid annotated to show the centre of rotation in the native glenohumeral joint;

FIGS. 4A and 4B show how the glenoid reference point may be determined with reference to Friedman's line;

FIG. 5 is an exploded perspective view of the implant components of a reverse implant;

FIGS. 6A and 6B show how the liner is attached to the patient's humerus, with FIG. 6A showing how the lateral offset of the liner may vary and with FIG. 6B showing how the humeral length may vary;

FIGS. 7A and 7B show a glenohumeral joint with a reverse implant with the arm relaxed in a 0 degrees orientation and with the arm raised to a 90 degrees orientation respectively;

FIGS. 8A and 8B are views of an implanted glenohumeral joint showing the effect of different implant geometries on the deltoid;

FIGS. 9A and 9B show two examples of how the glenosphere may be attached to the glenoid;

FIGS. 10A and 10B are details of the glenohumeral joint of FIGS. 6A and 6B respectively, annotated to indicate different offsets present in the joint when in the 0 and 90 degrees alignments;

FIG. 11 shows a first exemplary method of designing a reverse shoulder implant;

FIG. 12 shows an exemplary method of obtaining measurements from a native shoulder;

FIG. 13 shows an exemplary method of calculating the lateral offsets of the implanted shoulder 101 at 0 degrees and 90 degrees;

FIG. 14 shows a second exemplary method of designing a reverse shoulder implant;

FIG. 15 shows a third exemplary method of designing a reverse shoulder implant;

FIG. 16 shows a fourth exemplary method of designing a reverse shoulder implant.

### DETAILED DESCRIPTION

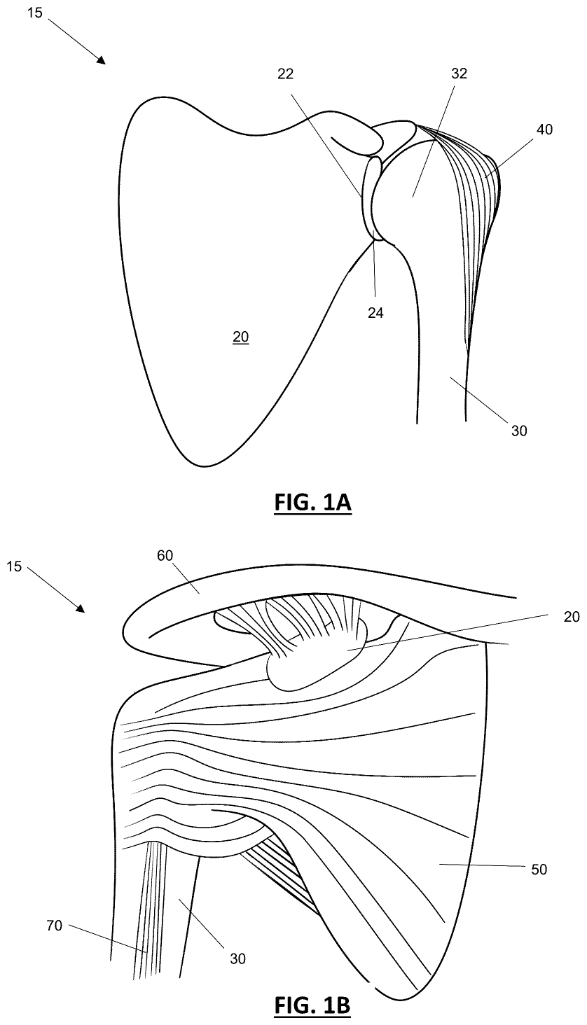

A healthy glenohumeral joint 15 is shown in FIG. 1A. The joint 15 includes the scapula 20 and the humerus 30. The humerus 30 terminates in a humeral head 32 that is received within the glenoid 22 formed in the scapula 20. The glenoid 22 presents a shallow dish for the humeral head 32, although the dish is deepened by the fibrocartilaginous glenoid labrum formed around the periphery of the glenoid 22 (not shown in FIG. 1A). The space 24 between the humeral head 32 and the glenoid 22 contains the synovial membrane and synovial fluid.

FIG. 1A also shows how the deltoid 40 joins to the scapula 20 and the humerus 30. The deltoid 40 also joins to the clavicle and acromion. The deltoid 40 assists in rotating the shoulder 15. The rotator cuff 50 also helps rotate the shoulder 15, and stabilises the shoulder 15. FIG. 1B shows how the parts of the rotator cuff 50 attach to the scapula 20 and the clavicle 60. FIG. 1B also shows part of the bicep 70 which attaches to the humerus 30 beneath the rotator cuff 50.

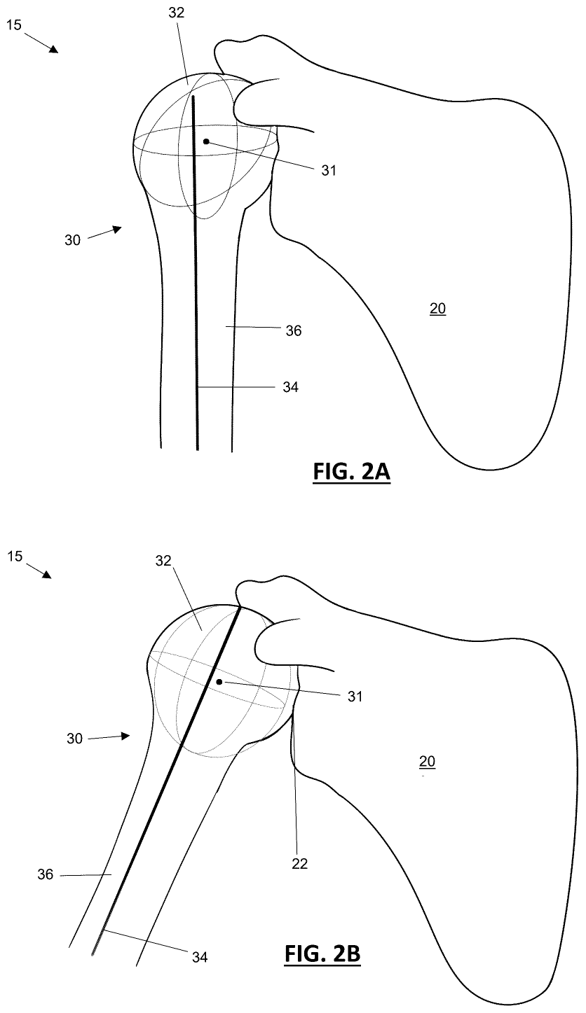

FIGS. 2A and 2B show a glenohumeral joint 15 with the arm relaxed in a 0 degrees orientation (FIG. 2A) and with the arm slightly raised in approximately 30 degrees orientation (FIG. 2B). FIGS. 2A and 2B also show the centre of the humeral head 31 and the humeral shaft axis 34 that extends along the length of the humerus 20 and can be used to define the orientation of the arm, i.e. 0 degrees orientation corresponds to the humeral shaft axis 34 being aligned vertically and 90 degrees orientation corresponds to the humeral shaft axis 34 being aligned horizontally. FIG. 2A includes three arrows that show directions commonly referred to when describing the shoulder 15.

Movement away from the torso is described as lateralisation (indicated as L in FIG. 2A), while movement towards the torso is described as medialisation (indicated as M), and movement downwards is described as inferiorisation (indicated as I).

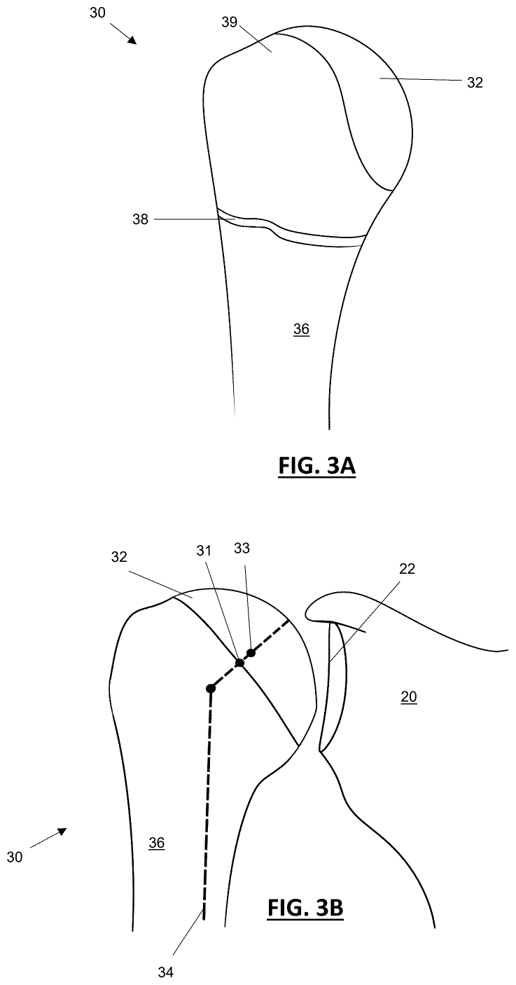

A more detailed representation of a native humeral head 32 is shown in FIG. 3A. The humerus 30 can see to comprise the humeral shaft 36 and the humeral head 32. FIG. 3A also shows the surgical neck 38 and the anatomical neck 39. FIG. 3B is a further view of the humeral 30 including the humeral head 32 and humeral shaft axis 34. The centre of the humeral head 32 is also shown as 31 which, as can be seen, is offset from the humeral shaft axis 34. In fact, the centre of rotation of the native shoulder is offset further still from the humeral shaft axis 34, as indicated at 33.

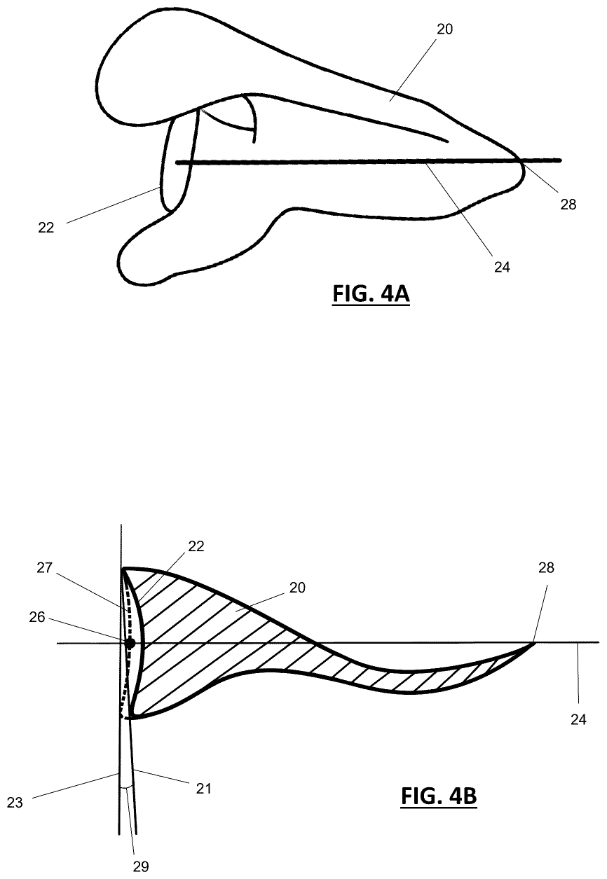

FIGS. 4A and 4B show how the glenoid reference point 26 may be determined with reference to Friedman's axis 24. Friedman's axis 24 is defined to correspond to line drawn along the long axis of the scapula 20 from the tip of the medial border 28 to the centre of the native glenoid 22 (or where the centre of the native glenoid 22 would be in an intact shoulder absent any bone erosion). FIG. 4B shows a native glenoid that has suffered from bone erosion, predominantly from the lower part of the glenoid. The solid line shows the actual native glenoid 22 whereas the dotted line 27 shows an approximation of the intact glenoid. The line 23 drawn across the intact glenoid 27 and the line drawn across the eroded glenoid 22 show the degree of bone loss that manifests as a version angle 29. The glenoid reference point 26 is the intersection of Friedman's axis 24 with intact glenoid 22 which, due to the definition of the Friedman axis, is where the centre of the intact glenoid would have been.

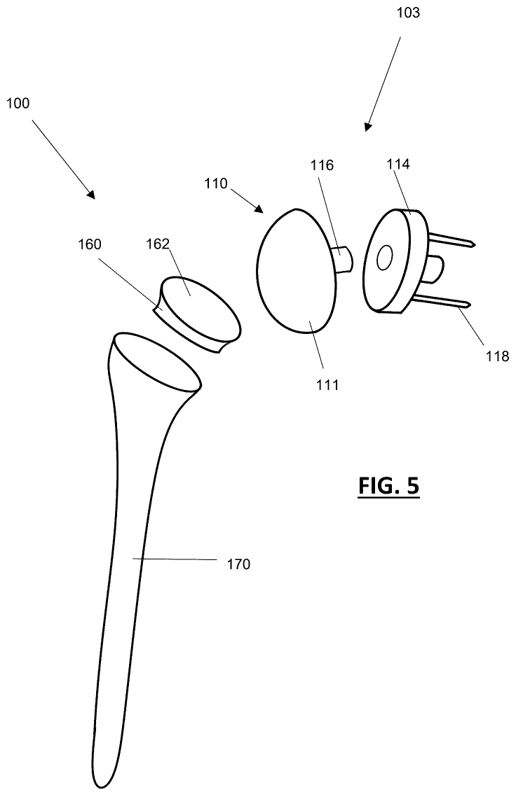

FIG. 5 is an exploded perspective view of the implant components 100 of a reverse implant. The implant components 100 include a glenosphere 110 and a liner 160. The glenosphere 110 comprises a half-sphere 112 that is received within a shallow dish 162 provided in the liner 160. The glenosphere 110 is part of a glenosphere assembly 103 that also includes a baseplate 114 and a lateralising peg 116. The glenosphere 110 attaches to the baseplate 114 via the lateralising peg 116. The baseplate 114 is fixed in place against the glenoid 130 with compression screws 118. The liner 160 is cemented to a humeral stem 170 that comprises a shaft 174 and a head 172. The liner 160 is attached to the head 172 of the humeral stem 170.

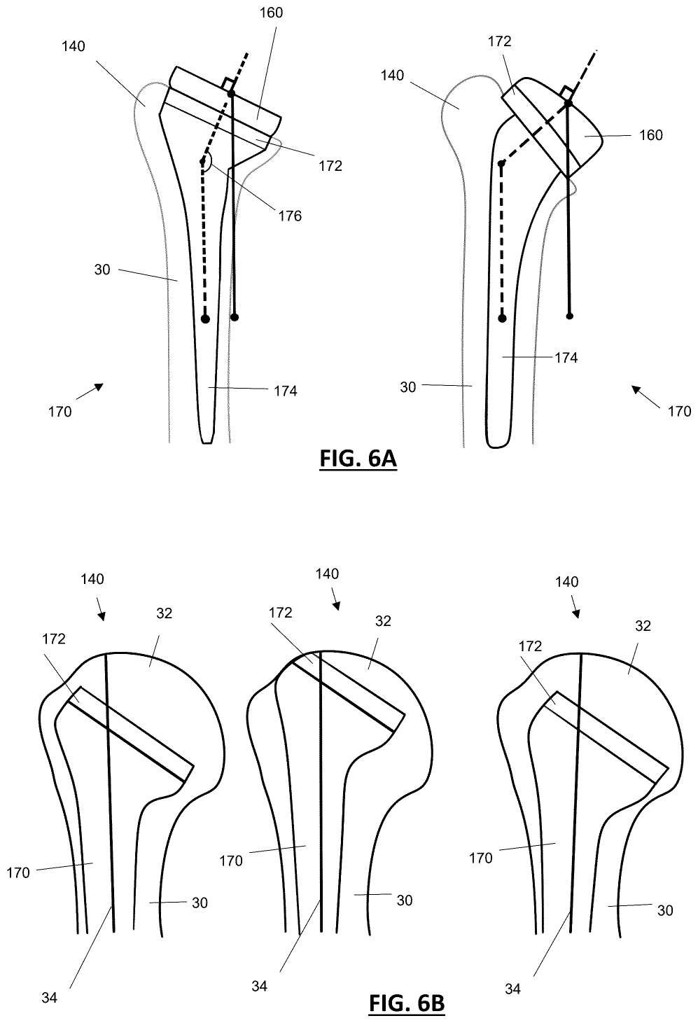

FIGS. 6A and 6B show how the humeral stem 170 and liner 160 attach to the patient's humerus 30. Before the humeral stem 170 can be fitted, the humerus 30 must be reshaped and hollowed out. The native humeral head 32 is removed by making a cut approximately in line with the anatomical neck 39. The condition of the native humerus 30 may mean that a higher cut may be better that leaves more of the humeral head 32, or a lower cut may be better that leaves less of the humerus 30. With the native humeral head 32 removed, the humerus 30 may be reamed to provide a cavity in which the shaft 174 of the humeral stem 174 is received and secured.

As can be seen from FIG. 6A, the front face of the head 172 of the humeral stem 170 is angled to match the angle of the cut made through the native humeral head 32. Thus, the liner 160 when seated in the humeral stem 170 is angled relative to the shaft 174 of the humeral stem 170. This is the neck angle 176 of the liner 160.

The humeral stem 170 may either be an inlay or an onlay. An example of an inlay is shown to the left in FIG. 6A where, as can be seen, the head 172 of the humeral stem 170 is received within the remodelled humerus 140. The right of FIG. 6B shows an onlay where the head 172 of the humeral stem 170 sits on top of the remodelled humerus 140. The onlay type of humeral stem 170 may be better suited to where more of the native humeral head 32 is lost when forming the remodelled humerus 140. FIG. 6B shows the length of the remodelled humerus 140 may vary depending on the where the cut is made across the native humeral head 32.

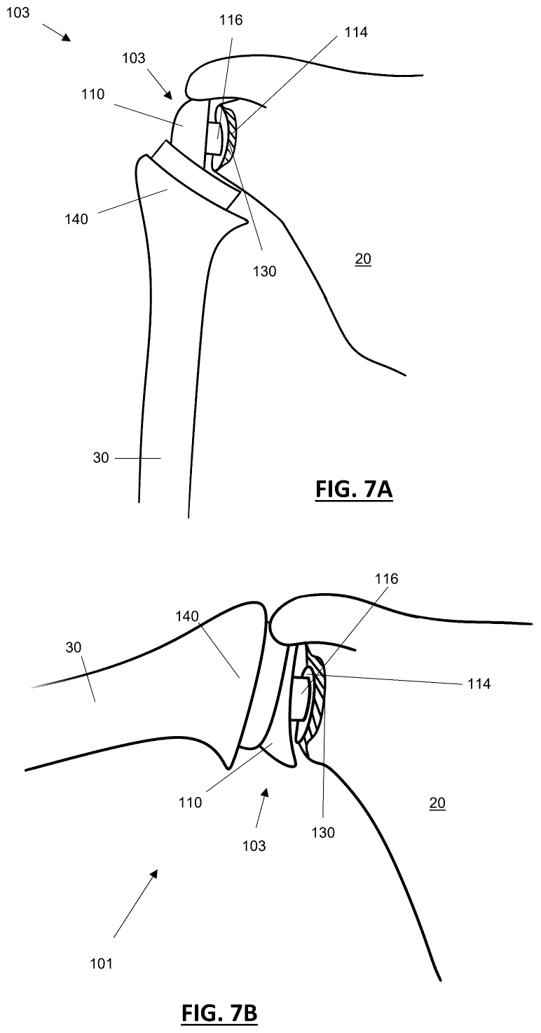

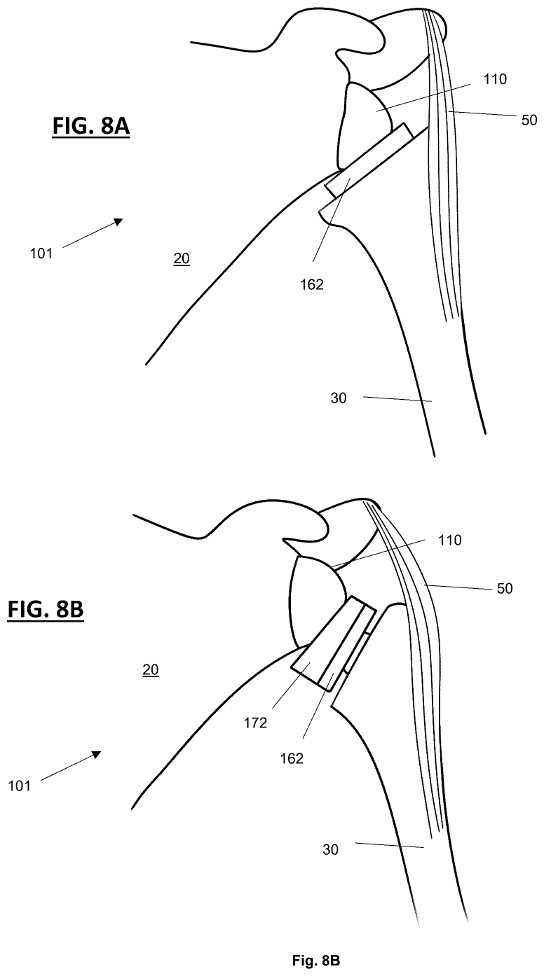

FIGS. 7A and 7B show a glenohumeral joint 101 with a reverse implant, with the arm relaxed in a 0 degrees orientation and with the arm raised to a 90 degrees orientation respectively. These figures show how the glenosphere assembly 103 attaches to the glenoid of the 130 patient. Generally, the native glenoid 22 will be worn and will require some remodelling to provide a good attachment point for the glenosphere assembly 101. FIGS. 7A and 8B show how the baseplate 114 is attached to the remodelled glenoid 130 with the compression screws 118. The lateralising peg 116 provides a lateral offset between the glenosphere 110 and the baseplate 114.

FIGS. 7A and 7B show how the liner 160 moves relative to the glenosphere 110 as the shoulder 101 moves between 0 degrees and 90 degrees orientations. As the deltoid 50 is tensioned and raises the arm to the 0 degrees position, the liner 60 pivots and slides across the glenosphere 110 as the deltoid 50 pulls up on the humerus 30. Thus, the centre of rotation 33 of the implanted shoulder 101 sits somewhere within the glenosphere 110.

FIGS. 8A and 8B shows the effects of differently designed reverse shoulder implants. FIG. 8A shows an “understuffed” reverse implant where the implanted shoulder 101 has insufficient lateralisation relative to the native shoulder 15. This leads to an insufficiently tensioned deltoid 50 that provides a relatively weak moment to rotate the implanted shoulder 101. An extremely understuffed shoulder 101 may result in insufficient force to stabilise the shoulder joint 101 such that the implanted shoulder 101 becomes susceptible dislocations. FIG. 8B shows an “overstuffed” shoulder 101 where the implanted shoulder 101 is overly lateralised relative to the native shoulder 15. This leads to over-tensioning in the deltoid 50, which is felt as stiffness in the implanted shoulder 101. Extremely overstuffed shoulders 101 can place so much strain on the bones to which the deltoid attaches that fractures occur.

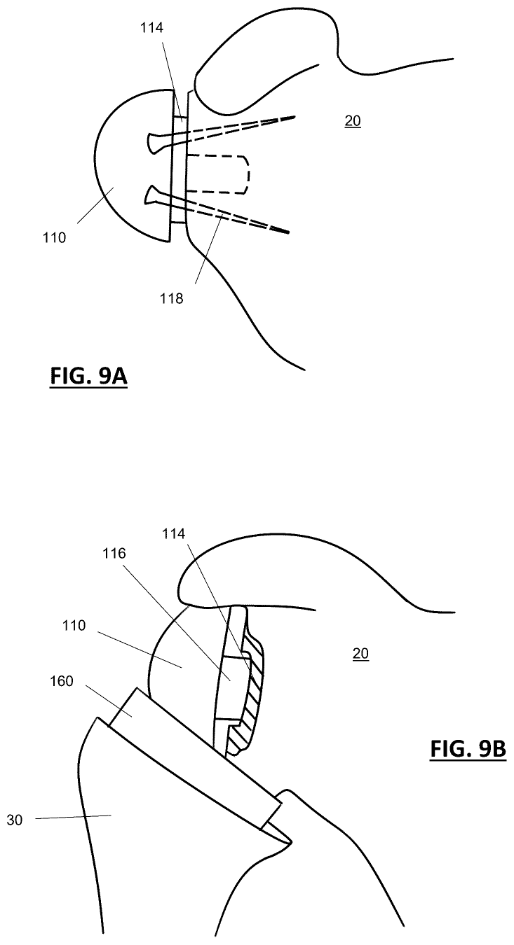

FIG. 9A shows one example of a glenosphere assembly 101 for use in a patient where there is little or no bone loss required for the remodelled glenoid 130. In this example, as little lateralisation of the shoulder 101 is required, a glenosphere 110 is attached directly to a baseplate 114 (i.e. without a lateralising a peg 116). FIG. 9B shows a second example of a glenosphere assembly 101, this time for use in a patient where there is more substantial bone loss in the remodelled glenoid 130. In this example, greater lateralisation of the shoulder 101 is required to return the centre of rotation 33 back to where it would lie in a healthy native shoulder 15. In this case, the glenosphere 110 is attached indirectly to the baseplate 114 via a lateralising a peg 116.

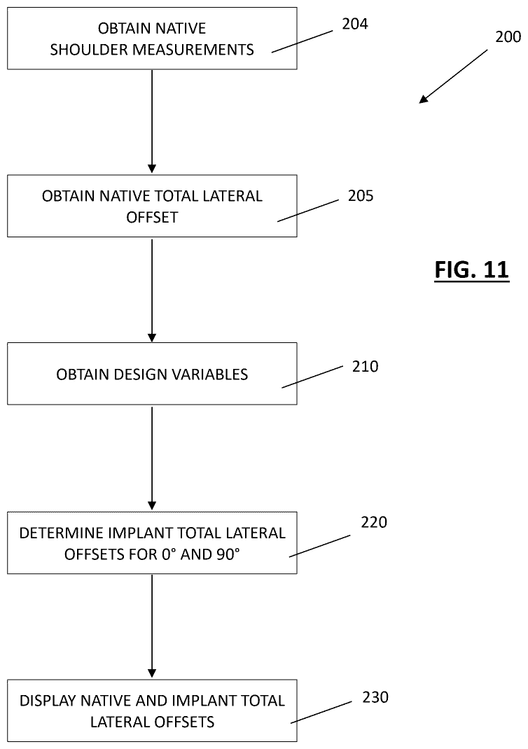

FIG. 11 shows a method 200 of designing a reverse shoulder implant. The method starts at step 204 where measurements of the native shoulder 15 are obtained. Then, at step 205, the total lateral offset for the native shoulder 15 is obtained from the native shoulder measurements. Then, at step 210, a set of design variables for the implant components 100 are obtained. At step 220, the design variables are used to determine the total lateral offset of the implanted shoulder 101 at 0 degrees and 90 degrees. At step 230, the total lateral offsets for the native shoulder 15 and for the implanted shoulder 101 at both 0 degrees and 90 degrees are displayed. This allows a person, such as a surgeon, to assess the contemplated implant and consider whether it is suitable or whether it should be improved.

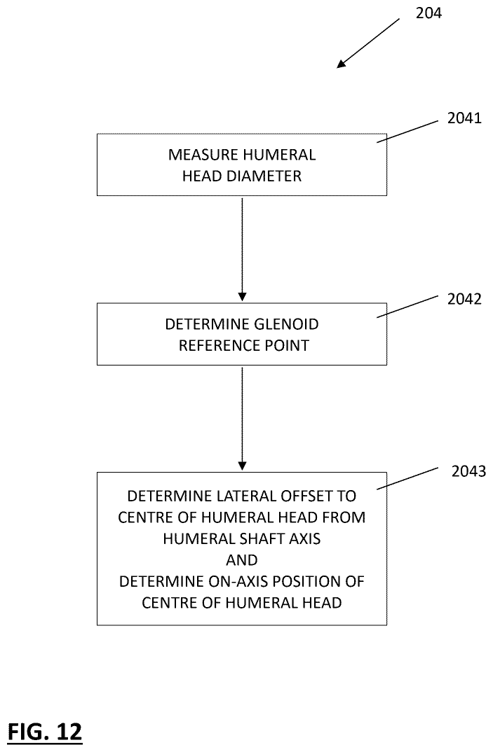

FIG. 12 shows an example implementation of step 204 in which the measurements of the native shoulder 15 are obtained. In the example of FIG. 12, the humeral head diameter is measured at step 2041, from a CT scan for instance. Step 2042 sees the glenoid reference point 26 determined. This may be performed using a scan of the native shoulder 15, and determined with reference to Friedman's axis 24 as has already been described. Step 2043 sees the lateral offset of the centre 31 of the humeral head 32 from the humeral shaft axis 34 determined. Step 2043 also sees the on-axis position of the centre 31 of the humeral head 32 determined (i.e. the position of the centre 31 of the humeral head 32 on the humeral shaft axis 34 from which the lateral offset of the centre 31 of the humeral head 32 is determined). As will be appreciated, steps 2041, 2042 and 2043 may be performed in any order.

The values determined in steps 2041, 2042 and 2043 may be used in the subsequent steps of methods 201, 202, 203 or 204. For example, determining the total lateral offset of the native shoulder 15 at step 205 may be performed using the diameter of the humeral head 32 found at step 2041. For example, the total lateral offset of the native shoulder 15 may be determined as 0.49 times the diameter found at step 2041 (i.e. 0.98 times the radius of the humeral head 32).

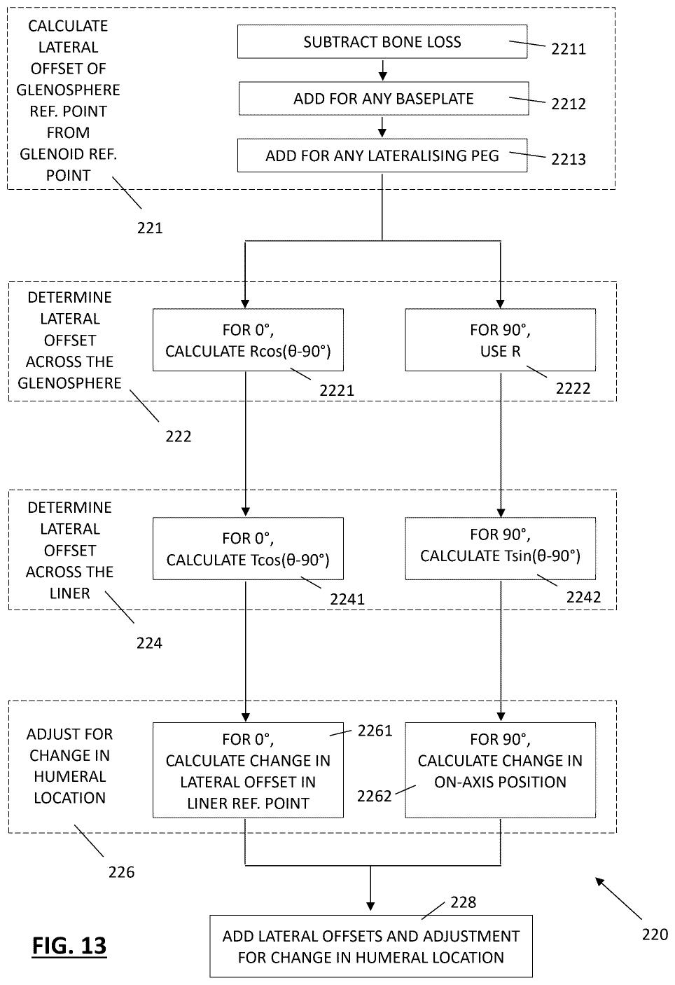

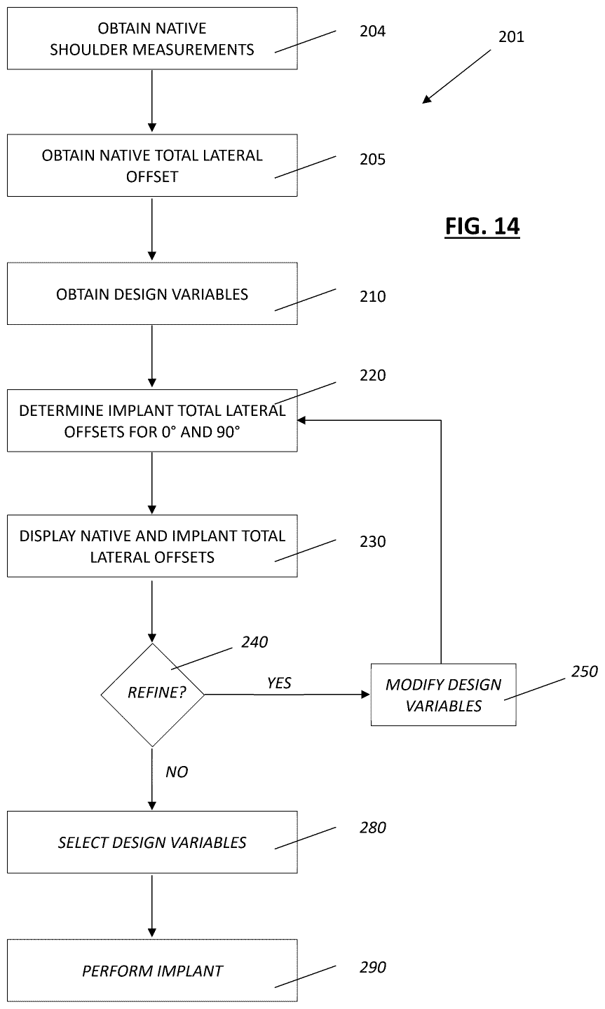

Also, the values determined in steps 2041, 2042 and 2043 may be used when obtaining the design variables of the implant components 100 at step 210. For example, the radius of the glenosphere 110 may be set to be equal to the radius of the native humeral head 32. Also, the lateral offset of the liner reference point 206 may be set to be equal to the lateral offset of the centre 31 of the humeral head 32 from the humeral shaft axis 34 determined at step 2043. The glenoid reference point 26 determined at step 2042 may be used when determining the lateral offsets of the implanted shoulder 101 at step 220, as will now be described in more detail with reference to FIG. 13. Moreover, the example implementation of step 204 shown in FIG. 12 and described above may be used in any of the example methods of FIGS. 14, 15 and 16.

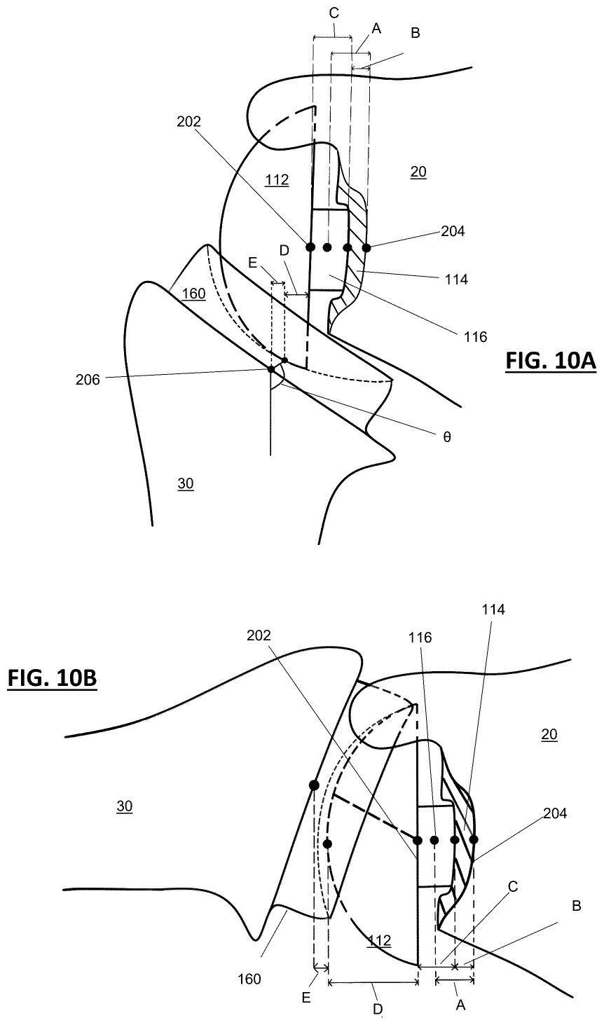

Step 220 begins at step 221 where the lateral offset of the glenosphere reference point 202 (see FIGS. 10A and 10B) from the glenoid reference point 26 is calculated. Step 221 comprises sub-steps 2211, 2212 and 2213. The glenosphere 110 comprises a part-sphere 112 and the glenosphere reference point 202 coincides with the centre of the corresponding complete sphere.

Calculating the lateral offset of the glenosphere reference point 202 from the native glenoid reference point 26 may comprise determining the lateral distance from the glenosphere reference point 202 to the attachment point 204 where the glenosphere 110 is attached to the bone of the glenoid 22 less the lateral length of any bone loss between the native glenoid reference point 26 and the attachment point 204. Hence, sub-step 2211 sees a subtraction of the length A shown in FIGS. 10A and 10B that is equal to the total bone loss. The distance from the glenosphere reference point 202 to the attachment point 204 may be calculated by adding the lateral thickness B of any baseplate 204 at sub-step 2212 and by adding the lateral length C of any lateralising peg 116 extending between the glenosphere reference point 202 and the attachment point 204 at sub-step 2213.

Step 222 sees the lateral offset D across the glenosphere 110 calculated. This is done differently for the 0 and 90 degree orientations. As shown sub-step 2221, for 0 degrees, the lateral offset D across the glenosphere 110 is calculated as Rcos(θ−90) where R is the radius of the glenosphere 110 and θ is the neck angle of the humeral stem 170. As shown at sub-step 2222, for 90 degrees the lateral offset D across the glenosphere 110 is determined to be R. Then, at step 224, the lateral offset E across the liner 160 is calculated.

This is the offset from the centre of the dish 162 of the liner 160 to the reference point 206 of the liner 160. The reference point 206 of the liner 160 is the point on the reverse side of the liner 160 directly behind the centre of the dish 162. Calculating the lateral offset E across the liner 160 is done differently for the 0 and 90 degree orientations. As shown sub-step 2241, for 0 degrees the lateral offset E across the liner 160 is calculated as Tcos(θ−90) where T is the thickness of the liner 160 measured from the centre of the dish 162 to the liner reference point 206. As shown sub-step 2241, for 90 degrees the lateral offset E across the liner 160 is calculated as Tsin(θ−90).

At step 226, an adjustment for a change in humeral location of the liner reference point 206 relative to the centre 31 of the humeral head 32 is calculated. As shown at sub-step 2261, for 0 degrees the adjustment is calculated as the difference in the lateral offset of the centre 31 of the humeral head 32 relative to the humeral shaft axis 34 and the lateral offset of the liner reference point 206 relative to the humeral shaft axis 34. As shown at sub-step 2262, for 90 degrees the adjustment is calculated as the change in the on-axis position of the liner reference point 206 relative to the on-axis position determined for the centre 31 of the humeral head 32.

Finally, at step 228 the total lateral offsets for 0 and 90 degrees are calculated by adding (1) the lateral offset C+B−A of the glenosphere reference point 202 from the glenoid reference point 26 calculated at step 221, (2) the lateral offset D across the glenosphere 110 calculated at step 222, (3) the lateral offset E across the liner 160 calculated at step 224, and (4) the adjustment for the change in humeral location of the liner reference point 206 relative to the centre of the humeral head 32 calculated at step 226.

This method of determining the total lateral offsets of the implanted shoulder 101 at 0 and 90 degrees may be used in any of the example methods of FIGS. 14, 15 and 16 that will now be described.

FIG. 14 shows a further method 201 of designing a reverse shoulder implant which is an extension of the method 200 of FIG. 11. Steps 202, 210, 220 and 230 are as for method 200 described above, and so will not be described again. After displaying the total lateral offsets for the native shoulder 15 and for the implanted shoulder 101 at both 0 degrees and 90 degrees at 230, the method 201 proceeds to step 240 where a decision is made as to whether the design variables should be refined. If the decision is not to refine the design variables, the method 201 proceeds to step 280 where the design variables of the implant components 100 are selected. The reverse implant may then be performed using implant components 100 with the selected design variables, as indicated at step 290.

However, if a decision is made at step 240 to refine the design variables, the method 201 continues to step 250 where one or more of the design variables are modified. The method then returns to step 220 where the modified set of design variables are used to determine the total lateral offset of the implanted shoulder 101 at 0 degrees and 90 degrees. At step 230, the total lateral offsets for the native shoulder 15 and for the implanted shoulder 101 at both 0 degrees and 90 degrees are displayed. The total lateral offsets for the implanted shoulder 101 obtained using the modified design variables are displayed in this instance. In addition, the total lateral offsets for the implanted shoulder 101 obtained using the original design variables may be displayed such that a comparison may be made between the outcomes using the original and modified design variables. This allows a determination as to whether the modified design variables have improved the implant. For example, a consideration of how close the 0 and 90 degrees total lateral offsets match the native total lateral offset may be made and/or how evenly the 0 and 90 degrees total lateral offsets are spread around the native total lateral offset.

The method then proceeds to step 240 once more where a further consideration is made as to whether the design variables should be refined further. If the decision is to refine the design variables further, the method continues to steps 250, 220 and 230 where the design variables are modified further, the new total lateral offsets for the implanted shoulder 101 at 0 and 90 degrees are determined, and the new total lateral offsets are displayed.

The method 201 continues looping through steps 250, 220, 230 and 240 each time a decision is made to refine the design variables still further. Once a decision is made not to refine the design variables further, the method continues to step 280, as per method 300 described above, where a set of design variables are selected.

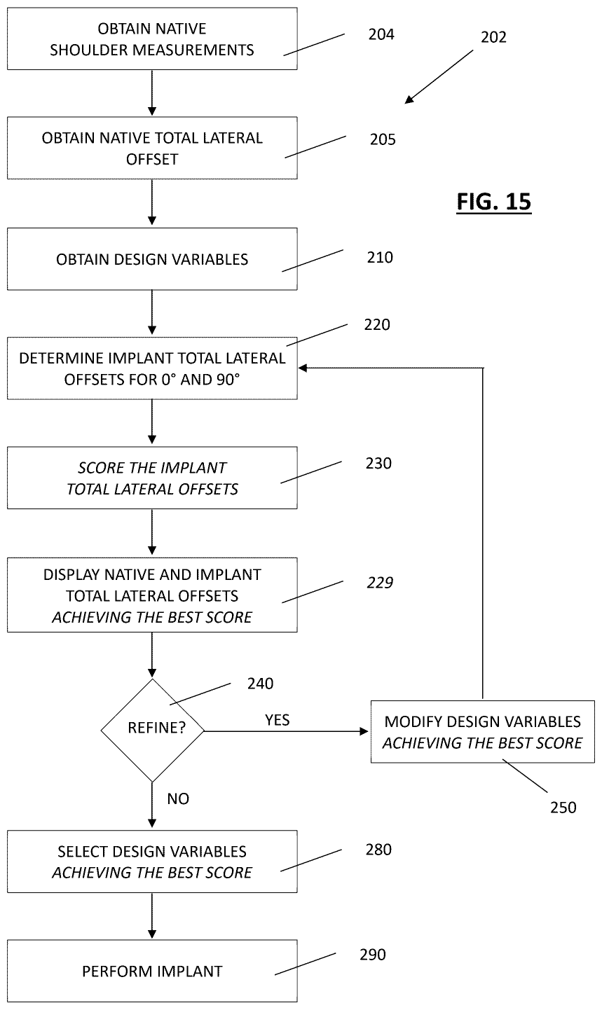

FIG. 15 shows an extension of the method 202 of FIG. 14 where assistance is provided in selecting a set of design variables. Method 202 of FIG. 15 includes a new step 229. After the total lateral offsets are determined for the implanted shoulder 101 at step 220, step 229 sees these total lateral offsets scored. This may be done in any number of ways. For example, the difference between the total lateral offset of the native shoulder 15 and the average value of the total later offsets of the implanted shoulder 101 may be used. Alternatively, the difference between the total lateral offsets of the implanted shoulder 101 may be used. A combination of these values may be used, for example a weighted combination.

Then, at step 230, the total lateral offset for the native shoulder 15 is displayed along with the total lateral offsets for the implanted shoulder 101 using the design variables of the implant components 100 achieving the best score. The method 202 proceeds to step 240 where, as before, a decision is made as to whether to refine the design variables further. If the determination is in the positive, step 250 sees a return to the design variables achieving the best score. For example, these design variables may be presented to a person for that person to use as a starting point for selecting one or more design variables for further modification.

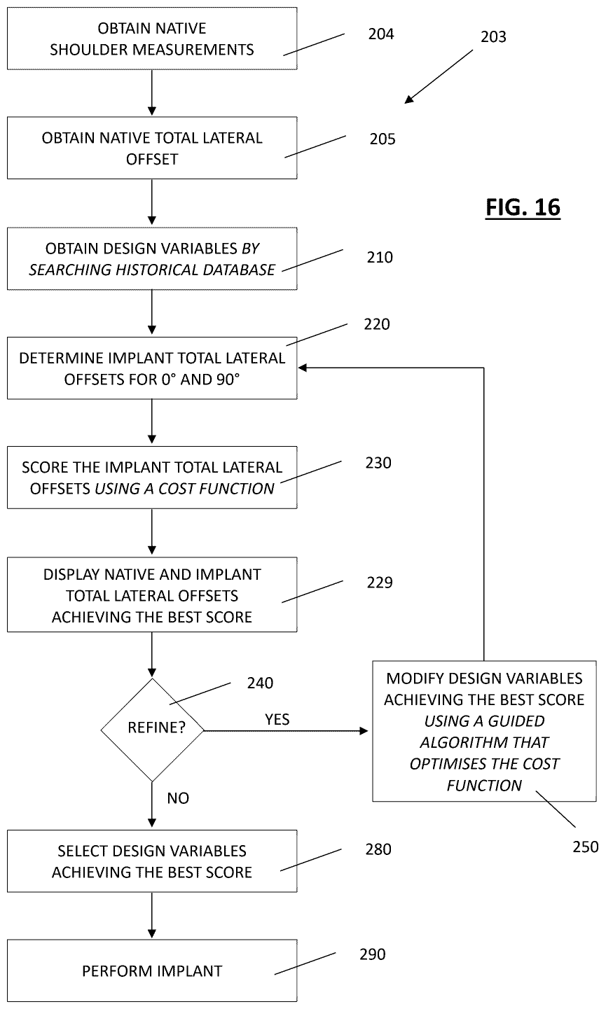

FIG. 16 shows another example method 203 of designing an implant featuring further automation. The method 203 broadly corresponds to the method 202 of FIG. 15, and so the following description focuses on the differences over the method 202 of FIG. 15.

Method 203 includes an automated way of obtaining the initial design variables of the implant components 100 the first time that step 210 is executed. Namely, a database of historical implant data is used. The database may contain records from past reverse shoulder implants. Each record may include measurements taken from the native shoulder 15, the design variables of the implant components 100 used in the implant, the total lateral offsets of the native shoulder 15 and implanted shoulder 101 and, optionally, a measure of the outcome of the reverse shoulder implant. Step 210 includes searching the records using the measurements of a native shoulder 15 that is to be fitted with a reverse implant. The records may be searched for the best match to the measurements of the native shoulder 15, and the corresponding design variables of the implant components 100 used in the implant that provides the best match may be identified and used as the design variables provided to step 220 the first time the lateral offsets of the implanted shoulder 101 are determined.

The search of the database of historical implant data may be performed in many different ways. For example, the best match of measurements of native shoulders 15 may be identified. Account may be taken of any measures of the outcome of the reverse shoulder implant. Different algorithms may be used to search the database, including the use of artificial intelligence/machine learning.

After the lateral offsets of the implanted shoulder 101 have been determined at step 220 using the design variables determined in accordance with step 210 as described immediately above, step 229 is used to score the lateral offsets of the implanted shoulder 101 relative to the lateral offset of the native shoulder 15. This may be performed using a cost function. The cost function may account for a number of results arising from the measurements of the native shoulder 15, the design variables of the implant components 100 and the calculated lateral offsets. For example, the cost function may score the total lateral offsets using a measure of the difference between the total lateral offsets of the implanted shoulder 101, as well as the difference between the total lateral offset of the native shoulder 15 and the average value of the total later offsets of the implanted shoulder 101.

Method 203 shown in FIG. 16 also includes an automated way of modifying the design variables at step 250. As explained above with reference to FIG. 15, step 250 selects the design variables achieving the best score for further modification. These design variables may then be further modified automatically, and the result on the cost function assessed to determine whether an improved set of design variables has been found. The design variables may be modified in a guided way, for example using a guided algorithm such as those that use local gradients to drive the cost function to a minimum value. The design variables may be modified in a random or pseudo-random way, for example by using genetic algorithm techniques. Hence, the cost function may be used to score different sets of design variables of the implant components at step 229 and to guide optimisation of the modified design variables at step 250.

A person skilled in the art will appreciate that the above embodiments may be varied in many different respects without departing from the scope of the present invention that is defined by the appended claims.

The order of steps within FIGS. 11 and 12 may be varied. For example, step 202 that sees the total lateral offset obtained for the native shoulder 15 need not be performed before steps 210 and 220 where the design variable and total lateral offsets for the implanted shoulder 101 are determined. Step 202 may be performed between steps 210 and 220, to concurrently with either or both of steps 210 and 220.

1. A method of designing a reverse shoulder implant comprising implant components that include a glenosphere for attachment to a patient's glenoid and a dished liner for attachment to a patient's humerus with the dish of the liner receiving the glenosphere, the method comprising:
determining a native total lateral offset of the native shoulder corresponding to the lateral offset of the centre of rotation position in the humeral head relative to a native glenoid reference point;
using a set of design variables of the implant components to determine a 0 degrees total lateral offset of the implanted shoulder corresponding to the lateral offset of a reference point on the liner from the native glenoid reference point when the implanted shoulder is in a 0 degrees alignment with the humeral shaft axis of the arm extending vertically;
using the set of design variables of the implant components to determine a 90 degrees total lateral offset of the implanted shoulder corresponding to the lateral offset of the reference point on the liner from the native glenoid reference point when the implanted shoulder is in a 90 degrees alignment with the humeral shaft axis extending horizontally; and
displaying the 0 degrees and 90 degrees total lateral offsets relative to the native total lateral offset.

2. The method according to claim 1, wherein determining the native total lateral offset comprises calculating the native total lateral offset to be the radius of the humeral head multiplied by a factor between 0.95 and 1 and, optionally, the method comprises measuring the radius of the native humeral head from an image taken of the patient's native shoulder.

3. The method according to claim 1, comprising determining the native glenoid reference point from an image taken of the patient's native shoulder and, optionally, wherein determining the native glenoid reference point comprises determining the point where Friedman's axis crosses the intact native glenoid.

4. The method according to claim 1, wherein:
the liner comprises a reverse side and an obverse side that is provided with the dish;
the liner reference point corresponds to where a line drawn perpendicularly through the centre of the dish intersects the reverse side; and
set of design variables includes the thickness of the liner which corresponds to the separation of the liner reference point from the centre of the dish.

5. The method according to claim 1, further comprising:
determining the 0 degrees total lateral offset to include an adjustment corresponding to the lateral offset of the liner reference point transverse to the humeral shaft axis relative to the lateral offset of the centre of the native humeral head transverse to the humeral shaft axis; and
determining the 90 degrees total lateral offset to include an adjustment corresponding to the on-axis offset of the liner reference point along the humeral shaft axis relative to the position of the centre of the native humeral head along the humeral shaft axis; and
wherein the set of design variables of the implant components include the lateral offset of the liner reference point transverse to the humeral shaft axis and the on-axis offset of the liner reference point along the humeral shaft axis.

6. The method according to claim 5, comprising determining the lateral offset of the centre of the native humeral head transverse to the humeral shaft axis from an image taken of the patient's native shoulder and, optionally, specifying the lateral offset of the liner reference point transverse to the humeral shaft axis to be equal to the lateral offset of the centre of the native humeral head transverse to the humeral shaft axis.

7. The method according to claim 1, wherein determining the 0 degrees and 90 degrees total lateral offsets of the implanted shoulder comprises:
determining the lateral offset of the centre of the dish of the liner from the native glenoid reference point;
determining the lateral offset across the liner; and
adding the lateral offset of the centre of the dish of the liner from the native glenoid reference point to the lateral offset across the liner.

8. The method according to claim 7, wherein determining the lateral offset of the centre of the dish of the liner from the native glenoid reference point comprises:
determining the lateral offset of a glenosphere reference point from the native glenoid reference point, wherein the glenosphere comprises a part-sphere and the glenosphere reference point coincides with the centre of the corresponding complete sphere;
determining the lateral offset across the glenosphere from the glenosphere reference point to the centre of the dish of the liner; and
adding the lateral offset of the glenosphere reference point from the native glenoid reference point to the lateral offset across the glenosphere.

9. The method according to claim 8, wherein:
determining the lateral offset of the glenosphere reference point from the native glenoid reference point comprises determining the lateral distance from the glenosphere reference point to the attachment point where the glenosphere is to be attached to the bone of the glenoid less the lateral length of bone loss between the native glenoid reference position and the attachment point, and wherein the set of design variables of the implant components includes the lateral length of bone loss between the native glenoid reference position and the attachment point; and, optionally,
the distance from the glenosphere reference point to the attachment point includes:
the lateral thickness of the baseplate if the glenosphere has a baseplate, the set of design variables of the implant components including the lateral thickness of the baseplate; and
the lateral length of the lateralising peg extending between the glenosphere reference point and the attachment point if the glenosphere is attached to the bone of the glenoid via a lateralising peg, the set of design variables of the implant components including the lateral length of the lateralising peg.

10. The method according to claim 8 wherein, when determining the 90degrees total lateral offset, determining the lateral offset across the glenosphere from the glenosphere reference point to the centre of the dish of the liner comprises calculating the lateral offset across the glenosphere as the radius of the glenosphere.

11. The method according to claim 8, wherein:
the set of design variables includes the radius of the glenosphere and the neck angle of the liner that is equal to the angle between the normal to the centre of the dish of the liner and the humeral shaft axis; and
when determining the 0 degrees total lateral offset, determining the lateral offset across the glenosphere from the glenosphere reference point to the centre of the dish of the liner comprises calculating the lateral offset across the glenosphere as the radius of the glenosphere times the cosine of a tilt angle equal to the neck angle minus 90 degrees and, optionally,
when determining the 0 degrees total lateral offset, determining the lateral offset across the liner comprises calculating the lateral offset across the liner as the thickness of the liner times the cosine of the tilt angle.

12. The method according to claim 11, wherein:
the set of design variables of the implant components includes a liner lateralisation value, and
when determining the 0 degrees total lateral offset, determining the lateral offset across the liner further comprises adding the liner lateralisation value to the calculated value of the thickness of the liner times the cosine of the tilt angle when determining the lateral offset across the liner at 0 degrees; and, optionally,
when determining the 90 degrees total lateral offset, determining the lateral offset across the liner comprises calculating the lateral offset across the liner as the thickness of the liner times the sine of the tilt angle.

13. The method according to claim 1, comprising specifying the glenosphere radius to be equal to the native humeral head radius and, optionally, measuring the native humeral head radius from an image taken of the patient's native shoulder.

14. The method according to claim 1, further comprising presenting the average of the 0 degrees and 90 degrees total lateral offsets relative to the native total lateral offset.

15. The method according to claim 1, wherein:
the set of design variables of the implant components is an original set of design variables of the implant components; and
the method further comprises:
generating an updated set of design variables of the implant components by modifying one or more of the design variables of the original set of design variables;
using the updated set of design variables to determine updated 0 degrees and 90 degrees total lateral offsets; and
presenting the updated 0 degrees and 90 degrees total lateral offsets relative to the native total lateral offset.

16. The method according to claim 15, wherein modifying one or more of the design variables comprises modifying one or more of the glenosphere radius, the thickness of the glenosphere baseplate, the lateral length of the lateralising peg extending between the glenosphere reference point and the attachment point, lateral length of bone loss between the native glenoid reference position and the attachment point, the thickness of the liner, the lateralisation of the liner, the lateral offset of the liner reference point transverse to the humeral shaft axis, the on-axis offset of the liner reference point along the humeral shaft axis, and the neck angle of the liner.

17. The method according to claim 15, further comprising:
presenting the average of the updated 0 degrees and 90 degrees total lateral offsets relative to the native total humeral offset; and/or
presenting the updated 0 degrees and 90 degrees total lateral offsets relative to the original 0 degrees and 90 degrees total lateral offsets.

18. The method according to claim 15, comprising:
one or more further iterations of the steps of generating updated sets of design variables of the implant components by modifying one or more of the design variables of the original set of design variables or an updated set of design variables;
using each updated set of design variables to determine updated 0 degrees and 90 degrees total lateral offsets; and
presenting each of the updated 0 degrees and 90 degrees total lateral offsets relative to the native total lateral offset.

19. The method according to claim 15, comprising selecting one set of design variables of the implant components from the initial set of design variables and the updated sets of design variables and, optionally, wherein selecting one set of design variables comprises selecting the set with the smallest difference between the average of the 0 degrees and 90 degrees total lateral offsets and the native total humeral offset and/or the smallest difference between the 0 degrees and 90 degrees total lateral offsets.

20. The method according to claim 15, wherein:
the design variables of the original set of design variables are set by a person; or
the design variables of the original set of design variables are determined by a computer system using an analysis of historical data providing design variables for previously performed reverse shoulder implants.

21. The method according to claim 15, wherein:
the design variables of the updated sets of design variables are set by a person; or
the design variables of the updated sets of design variables are determined by a computer system using a brute force or guided algorithm method, optionally by a computer system selecting one set of design variables using a cost function to evaluate the original 0 degrees and 90 degrees total lateral offsets relative to the native total lateral offset and the updated 0 degrees and 90 degrees total lateral offsets relative to the native total lateral offset.

22. A computer program comprising computer program instructions that, when executed by one or more computer processors, cause the one of more computer processors to perform the method of claim 1, or a computer readable medium having such a computer program stored thereon.

23. A computer system comprising one or more computer processors and a computer memory having stored therein a computer program comprising computer program instructions that, when executed by the one or more computer processors, cause the one of more computer processors to perform the method of claim 1.

24. A set of implant components configured for a reverse shoulder implant comprising:
a glenosphere assembly including a glenosphere and a baseplate for attaching the glenosphere to the glenoid of a patient;
a humeral stem for implanting into the humerus of the patient; and
a dished liner fastened to the humeral stem; and
wherein the design variables of the set of implant components are such that the 0 degrees and 90 degrees total lateral offsets of the implanted shoulder match the total lateral offset in the patient's native shoulder, and wherein:
the total lateral offset of the native shoulder corresponds to the lateral offset of the centre of rotation position in the native humeral head relative to a native glenoid reference point;
the 0 degrees total lateral offset of the implanted shoulder corresponds to the lateral offset of a reference point on the liner from the native glenoid reference point when the implanted shoulder is in a 0 degrees alignment with the humeral shaft axis of the arm extending vertically; and
the 90 degrees total lateral offset of the implanted shoulder corresponding to the lateral offset of the reference point on the liner from the native glenoid reference point when the implanted shoulder is in a 90 degrees alignment with the humeral shaft axis extending horizontally.
